(19) United States
(12) Patent Application Publication
(10) Pub. No.: US 2007/0190039 A1
(43) Pub. Date: Aug. 16, 2007

(54) Novel cell lines, and methods of preparation and use thereof

(76) Inventor: Probal Banerjee, Staten Island, NY (US)

Correspondence Address: COZEN O'CONNOR, P. C., 1900 MARKET STREET, PHILIADELPHIA, PA, 19103-3508, US

(21) Appl. No.: 10/941,478

(22) Filed: Sep. 14, 2004

Related U.S. Application Data
(60) Provisional application No. 60/503,211, filed on Sep. 15, 2003.

(51) Int. Cl. A61K 48/00; C12N 5/06; C12N 5/08; C12N 15/09

(52) U.S. Cl. 424/93.21; 435/455; 435/368; 435/354

(57) ABSTRACT

The present invention provides, in some embodiments, hippocampal neuron-derived cell lines that display voltage-gated calcium channels of various types, including the N-type channels. Such mixed calcium channel activity is similar to that observed in the hippocampal neurons from which they were originally derived. The cells also can be differentiated into neurons and contain many of the important hippocampal neuron proteins. The invention further provides methods for using the cells.

### CROSS REFERENCE TO RELATED APPLICATIONS

This application claims priority benefit of U.S. Provisional Application Ser. No. 60/503,211, filed Sep. 15, 2003, the entire content of which is incorporated by reference herein.

### FIELD OF THE INVENTION

The present invention is directed to novel cell lines that display significant levels of voltage-gated calcium current, and to methods for their preparation and use.

### BACKGROUND OF THE INVENTION

Voltage-gated calcium channels are a fundamental requirement of the nerve cells, as they are essential for the generation of the typical electrical activity of the neurons, termed “action potential”. Action potentials are required for the release of small molecules called neurotransmitters from the nerve terminals. These neurotransmitters then bind to specific receptor molecules on other neurons and also muscle cells. This triggers appropriate signaling cascades that eventually lead to various biological effects such as memory formation in the brain, or an action in limbs. Thus, voltage-gated calcium channels are central to signaling by the nerve cells.

Impairment of such signaling causes many of the commonly known behavioral defects, such as Depression and Anxiety, and also impairment of movement, as observed in Myasthenia Gravis, Parkinson's disease, Muscular Dystrophy, and other central nervous system (CNS) diseases and disorders. Such conditions are often caused by alterations of signals that modulate the activity of these voltage-gated calcium channels, but the mechanism of such signal modulation is far from clear.

In order to perform research to understand intraneuronal signaling cascades that regulate voltage-gated ion channels, cell lines are required that mimic the action of neurons. Although neurons can be isolated from the brain of prenatal mice and cultured under known conditions, these cells do not multiply and have to be used up once they are isolated from prenatal mice. They are also in a limited quantity, which makes their utilization in research difficult. To avoid dissection of embryonic mice every time such immortalized neurons are required, such cells can be immortalized by fusing them to neurotumor cells that multiply indefinitely. The result after appropriate selection is hybrid cells that have neuronal properties and can also divide to yield many cells. Many such neuronal cell lines are currently available. They can be chemically induced (by for example differentiation) to stop dividing, and to acquire most properties of a neuron. However, the hybridization process which makes such cell lines could also turn off the synthesis of proteins that associate to form the calcium channels. At the present time, none of the cell lines derived from the brain neurons display a neuron-specific, voltage-gated N-type calcium channel.

The hippocampus is a horse-shoe-like midbrain formation which is densely packed with layers of specialized neurons, that play an important role in both short-term and long-term memory formation. Given the importance of understanding the mechanisms of both memory, and the diseases and disorders that affect memory, it can be seen that a cell line that provides a model of hippocampal neurons would be great importance for studying the signaling cascades involved in memory formation, and for discovery of potential therapeutics for treating such diseases and disorders. This invention is directed to these, as well as other, important ends.

### SUMMARY OF THE INVENTION

In one aspect, the present invention provides methods for inducing voltage gated calcium current activity in a mammalian hippocampal neuron-derived cell comprising the steps of providing a mammalian hippocampal neuron-derived cell; and transfecting the cell with a recombinant construct comprising a nucleotide sequence encoding an ATPase, which in some embodiments is a P-type ATPase, and which in some embodiments is an ATPase II.

In some embodiments, the mammalian hippocampal neuron-derived cell is a murine derived cell. In some embodiments, the mammalian hippocampal neuron-derived cell is a HN2 cell.

In some embodiments, the recombinant construct comprises a nucleotide sequence encoding a mammalian ATPase II. In some embodiments, the nucleotide sequence encodes a murine ATPase II. In some embodiments, the nucleotide sequence encoding the murine ATPase II comprises the sequence of GeneBank accession number U75321. In some embodiments, the recombinant construct comprises the expression vector pCMV6c.

In a further aspect, the present invention provides methods for identifying a compound that inhibits calcium channels in hippocampal neurons comprising the steps of contacting one or more candidate compounds with an ATPase-transformed mammalian hippocampal neuron-derived cell; and measuring the ability of the compound or compounds to inhibit activity of said channels. In some embodiments, the ATPase-transformed mammalian hippocampal neuron-derived cell is an ATPase II-transformed mammalian hippocampal neuron-derived cell. In some embodiments, the ATPase-transformed mammalian hippocampal neuron-derived cell comprises N-type calcium channel activity.

In some embodiments, the ATPase-transformed mammalian hippocampal neuron-derived cell is a murine derived cell. In further embodiments, the ATPase-transformed mammalian hippocampal neuron-derived cell is derived from a HN2 cell. In some embodiments, the ATPase-transformed mammalian hippocampal neuron-derived cell is produced by a process comprising the steps of a) providing a mammalian hippocampal neuron-derived cell; and b) transfecting said cell with a recombinant construct comprising a nucleotide sequence encoding an ATPase, which in some embodiments is a P-type ATPase, and in some embodiments is an ATPase II.

In some embodiments, the recombinant construct comprises a nucleotide sequence encoding a mammalian ATPase II. In some embodiments, the recombinant construct comprises a nucleotide sequence encoding a murine ATPase II. In further embodiments, the nucleotide sequence encoding said murine ATPase II comprises the sequence of GeneBank accession number U75321. In some embodiments, the recombinant construct comprises the expression vector pCMV6c.

In a further aspect, the present invention provides methods for identifying a compound that stimulates a receptor that inhibits calcium channels in hippocampal neurons comprising the steps of contacting one or more candidate compounds with an ATPase transformed mammalian hippocampal neuron-derived cell, wherein said cell expresses at least one receptor that modulates said calcium channels; and measuring the ability of the compound or compounds to inhibit activity of said channels.

In some embodiments, the ATPase-transformed mammalian hippocampal neuron-derived cell is a P-type ATPase-transformed mammalian hippocampal neuron-derived cell. In some embodiments, the ATPase-transformed mammalian hippocampal neuron-derived cell is an ATPase II-transformed mammalian hippocampal neuron-derived cell.

In some embodiments, the ATPase transformed mammalian hippocampal neuron-derived cell comprises N-type calcium channel activity. In further embodiments, the ATPase transformed mammalian hippocampal neuron-derived cell is a murine derived cell. In further embodiments, the ATPase transformed mammalian hippocampal neuron-derived cell is derived from a HN2 cell. In some embodiments, the ATPase transformed mammalian hippocampal neuron-derived cell is produced by a method comprising the steps of a) providing a mammalian hippocampal neuron-derived cell; and b) transfecting said cell with a recombinant construct comprising a nucleotide sequence encoding an ATPase, or a P-type ATPase, or an ATPase II. In some embodiments, the methods further comprise the step of (c) transfecting said cell with a recombinant construct comprising a nucleotide sequence encoding a receptor that modulates said calcium channels. In some embodiments, the recombinant construct of said step (c) comprises a nucleotide sequence encoding a receptor selected from purinergic receptors, serotonin 1A receptors, alpha-adrenergic receptors, adenosine receptors, opiodergic receptors (for example the kappa opioid receptor) and somatostatin receptors. Other suitable receptors will be apparent to those of skill in the art.

In some embodiments, the transfections in steps (b) and (c) are performed simultaneously, for example by using a single recombinant construct. In some embodiments, the transfections in steps (b) and (c) are performed separately. In some embodiments, the transfections in steps (b) and (c) are performed contemporaneously, using different expression vectors.

In some embodiments, the recombinant construct comprises a nucleotide sequence encoding a mammalian ATPase II. In further embodiments, the recombinant construct comprises a nucleotide sequence encoding a murine ATPase II. In further embodiments, the nucleotide sequence encoding said murine ATPase II comprises the sequence of GeneBank accession number U75321. In some embodiments, the recombinant construct comprises the expression vector pCMV6c.

In some embodiments of the foregoing methods, the ATPase transformed mammalian hippocampal neuron-derived cell expresses a receptor selected from purinergic receptors, serotonin 1A receptors, alpha-adrenergic receptors, adenosine receptors, opiodergic receptors and somatostatin receptors.

The present invention further provides compounds that inhibits calcium channels in hippocampal neurons identified by the method of the invention, and compounds that stimulate receptors that inhibit calcium channels in hippocampal neurons identified by the method of the invention.

In some further embodiments, the present invention provides methods for treating a disease or disorder, or a symptom thereof, that is characterized by deleterious calcium channel activity comprising administering to an individual in need thereof a therapeutically effective amount of a compound identified by one or more the methods of the invention disclosed herein. In some embodiments, the disease or disorder is pain management, ischemic attack, stroke, depression, anxiety, and impairment of movement, as observed for example in Myasthenia Gravis, Parkinson's disease, Muscular Dystrophy, and other central nervous system (CNS) diseases and disorders.

In a further aspect, the present invention provides ATPase transformed mammalian hippocampal neuron-derived cells having voltage gated calcium current activity, P-type ATPase transformed mammalian hippocampal neuron-derived cells having voltage gated calcium current activity, and ATPase II transformed mammalian hippocampal neuron-derived cells having voltage gated calcium current activity. In some embodiments, the foregoing transformed mammalian hippocampal neuron-derived cells possess N-type calcium channel activity. In some embodiments, the foregoing transformed mammalian hippocampal neuron-derived cells possess N-type calcium channels.

In some embodiments, the ATPase transformed mammalian hippocampal neuron-derived cells are produced by a method comprising transfecting a mammalian hippocampal neuron-derived cell with a recombinant construct comprising a nucleotide sequence encoding an ATPase II. In some embodiments, the mammalian hippocampal neuron-derived cell is a murine derived cell. In further embodiments, the mammalian hippocampal neuron-derived cell is a HN2 cell.

In some embodiments of the ATPase transformed mammalian hippocampal neuron-derived cells, the recombinant construct comprises a nucleotide sequence encoding a mammalian ATPase II. In further embodiments the recombinant construct comprises a nucleotide sequence encoding a murine ATPase II. In some embodiments, the nucleotide sequence encoding the murine ATPase II comprises the sequence of GeneBank accession number U75321. In some embodiments, the recombinant construct comprises the expression vector pCMV6c.

In some embodiments, the invention provides an ATPase II transformed mammalian hippocampal neuron-derived cell having voltage gated calcium current activity produced by a method comprising transfecting a HN2 cell with a recombinant construct comprising an expression vector; said expression vector comprising a nucleotide sequence encoding a murine ATPase II; wherein said nucleotide sequence has the sequence of GeneBank accession number U75321. In some such embodiments, the recombinant construct comprises the expression vector pCMV6c.

The present also provides the novel cell lines HN2A12 (ATTC Accession # PTA-5440) and HN2A22 (ATTC Accession # PTA-5441).

### BRIEF DESCRIPTION OF THE DRAWINGS

FIG. 1 shows the appearance of voltage-gated calcium current in clones overexpressing ATPase II.

FIG. 2 shows the effect of CdCl₂ and ω-conotoxin on voltage-gated calcium current.

FIG. 3 shows the increased expression of the α-_{1B} subunit of the N-type calcium channels in HN2A12 and HN2A22 cells.

### DETAILED DESCRIPTION

It has been discovered in accordance with the present invention that cells possessing voltage-gated calcium channels of various types, including for example the N-type channels, can be prepared by transfecting a mammalian hippocampal neuron-derived cell with a construct that includes a nucleotide sequence encoding an ATPase, which in some embodiments is a P-type ATPase, or an ATPase II, preferably a mammalian ATPase II. In some embodiments, the construct contains a mammalian APTase II cDNA. The cells possess mixed calcium channel activity similar to that observed in the hippocampal neurons from which the cells are derived.

In one aspect, the present invention provides methods for inducing voltage gated calcium current activity in a mammalian hippocampal neuron-derived cell comprising the steps of providing a mammalian hippocampal neuron-derived cell; and transfecting the cell with a recombinant construct comprising a nucleotide sequence encoding an ATPase, which can be a P-type ATPase, or an ATPase II.

In a further aspect, the present invention provides methods for identifying a compound that inhibits calcium channels in hippocampal neurons comprising the steps of contacting one or more candidate compounds with an ATPase-transformed mammalian hippocampal neuron-derived cell; and measuring the ability of the compound or compounds to inhibit activity of said channels.

In a further aspect, the present invention provides methods for identifying a compound that stimulates a receptor that inhibits calcium channels in hippocampal neurons comprising the steps of contacting one or more candidate compounds with an ATPase transformed mammalian hippocampal neuron-derived cell, wherein said cell expresses at least one receptor that modulates said calcium channels; and measuring the ability of the compound or compounds to inhibit activity of said channels.

In some embodiments of the methods and compounds of the invention, the ATPase-transformed mammalian hippocampal neuron-derived cell comprises N-type calcium channel activity.

The mammalian hippocampal neuron-derived cell can be a hybrid neuroblastoma that can be derived from a wide variety of mammalian sources. For example and not limitation, the mammalian hippocampal neuron-derived cell can be a murine-derived hybrid neuroblastoma cell. Examples of such suitable cells are HN2 cells and HN33 cells (see Lee, H. J., et al., (1990) J. Neurosci., 10, 1779-1787, incorporated herein by reference for all purposes. Other suitable cells will be apparent to those of skill in the art.

In accordance with some embodiments of the invention, a mammalian hippocampal neuron-derived cell is transfected with a recombinant construct that includes a nucleotide sequence that encodes an ATPase II. An example of such a recombinant construct is an expression vector suitable for transfection and expression of proteins in mammalian cells. Any of the wide variety of such expression vectors are amendable to the methods and cells of the invention. Exemplary expression vectors can be found in the 2003 catalogs from Promega Corporation (Madison, Wis.) and BD Biosciences (Clontech), which are incorporated by reference in their entirety for all purposes. One such suitable expression vector is pCMV6c. Other suitable vectors will be apparent to those of skill in the art.

Transfection of the expression vector can be performed by a variety of methods as are known in the art. Such methods include electroporation and transfection using lipid-based DNA delivery systems, for example Effectene and Superfect (Qiagen, Valencia, Calif.).

Methodologies for performing such transfections are well known in the art. Exemplary protocols for such transfections can be found Sambrook, J., and Russel, D. W. “Molecular Cloning: A Laboratory Manual” January 2001, which is incorporated by reference in its entirety for all purposes.

A wide variety of ATPase nucleotide sequences can be employed in the methods and cells of the invention. Examples of a suitable ATPase sequences include ATPase II sequences, for example those of murine ATPase II, for example the sequence of GeneBank accession number U75321.

The ATPase transformed cells of the invention provide a model for hippocampal neuron activity that displays calcium channel activity similar to that of hippocampal neurons. Thus, the ATPase transformed cells of the invention are useful for the identification of compounds that modulate such calcium channel activity, including therapeutics for use in the treatment of diseases and disorders, or the amelioration of a symptom of such a disease or disorder, that is characterized by, or which involves abnormal calcium channel activity.

Thus, in accordance with the present invention, methods are provided for identifying compounds that inhibit calcium channels in hippocampal neurons comprising the steps of contacting one or more candidate compounds with an ATPase-transformed mammalian hippocampal neuron-derived cell; and measuring the ability of the compound or compounds to inhibit activity of said channels.

In further embodiments, methods are provided for treating diseases and/or disorders, and for the amelioration of symptoms of such disease and/or disorders, that include administering a compound identified by one or more methods of the invention to an individual (for example mammal) in need thereof.

In accordance with the methods of the invention, the cells of invention can be used to identify compounds that exert their effect directly on calcium channels, or compounds that act through interaction with one or more receptors that in turn modulate such calcium channels. For example, the cell lines HN2A12 and HN2A22 described herein possess the opoid receptors and prostaglandin receptors of the hippocampal cells from which they were derived. Thus, these cells can be used to identify compounds that modulate calcium channel activity by interaction with these receptors.

A number of receptors are involved in the regulation of voltage-gated calcium channels. These include several receptors that regulate the activity of brain neurons, for example the purinergic receptors (See J. Neurochem. (2002) 83, 285-298; J. Pharmacol. Exp. Ther. (2002) 303, 520-526) and the serotonin 1A receptors (See J. Neurosci. (2002) 22, 1248-1255, Synapse (2001) 41, 351-359. Several such receptors also regulate the activity of both brain neurons and cardiac cells. Examples of such receptors include the alpha-adrenergic receptors (See Eur. J. Neurosci. (2003) 17, 1411-1424) and the adenosine receptors (See J. Pharmacol. Exp. Ther (2001) 299, 501-508. Other such receptors regulate the activity of both brain cells and sympathetic neurons. Examples of these receptors include the opiodergic receptors [See J. Neurochem. (2003) 85, 34-39, and Brain Res. (2002), 942, 11-22] and the somatostatin receptors [See J. Neurosci. (2002) 22, 5374-5386. J. Physiol. (2001) 535, 519-532].

Calcium channel regulation receptors, including but not limited to the receptors named above, are crucial for normal function of the neurons in the brain, cardiac and pituitary cells and also the sympathetic and peripheral neurons in the pain pathway. In some embodiments, the ATPase transformed cells of the invention can further include one or more such receptors that are not expressed in ATPase transformed cell (or that are only expressed in low amounts therein). Such cells can be prepared, for example, by transfection with an expression vector containing a nucleic acid for a receptor that directly or indirectly modulate calcium channel activity. Examples of such receptors include purinergic receptors, serotonin 1A receptors, alpha-adrenergic receptors, adenosine receptors, opiodergic receptors and somatostatin receptors. In some embodiments, the receptor of interest is transfected into the ATPase transformed cell. In further embodiments, the nucleic acid of a desired receptor is included within the expression vector used to introduce the APTase DNA into the mammalian hippocampal neuron-derived cell, or placed in different expression vector that are cotransfected into the same cells.

Thus, in some embodiments, the present invention provides mammalian hippocampal neuron-derived cell lines that express voltage-gated calcium channels, and that also express a receptor that is involved in the regulation of such voltage-gated calcium channels. In some embodiments, the cells are prepared by transfecting a recombinant mammalian hippocampal neuron-derived cell of the invention with a nucleic acid, for example a cDNA (via an appropriate expression vector as is known in the art), that encodes a receptor that is involved in the regulation of voltage-gated calcium channels. In some embodiments, the receptor is chosen from among those described above, although all such receptors are within the scope of the invention.

Thus, in a further aspect, the present invention provides methods for identifying a compound that stimulates a receptor that inhibits calcium channels in hippocampal neurons comprising the steps of contacting one or more candidate compounds with an ATPase transformed mammalian hippocampal neuron-derived cell, wherein said cell expresses at least one receptor that modulates said calcium channels; and measuring the ability of the compound or compounds to inhibit activity of said channels.

In some embodiments, the present invention provides two hippocampal neuron-derived cell lines (designated HN2A12 and HN2A22) that display voltage-gated calcium channels of various types, including the N-type channels. Such mixed calcium channel activity is similar to that observed in the hippocampal neurons from which they were originally derived. These cells also can be differentiated into neurons and contain many of the important proteins that hippocampal neurons harbor, e.g. the opoid receptors, the prostaglandin receptors, and proteins called G-proteins.

Compounds identified by the methods of the invention can be used in the treatment of diseases and disorders (and in the amelioration of symptoms of diseases and disorders), that are characterized by deleterious levels of calcium channel activity. For example, it is known that ischemic attack (for example during myocardial infarction) elicits a large increase in calcium channel activity, which is highly detrimental to the patient. Compounds identified by the methods disclosed herein can be used therapeutically to stimulate receptors that will inhibit the deleterious calcium channel activity, thus preventing or ameliorating damage from the ischemic attack. For example, the HN2A12 and HN2A22 cells of the invention express opiodergic receptors. Thus, in some embodiments of the invention, these cells are used in screening assays to determine the efficacy of candidate compounds for modulation of calcium channel activity by modulation of opiodergic receptor activity.

Further examples of diseases and/or disorders amenable to the methods of the invention include pain management, ischemic attack, stroke, depression, anxiety, and impairment of movement, as observed for example in Myasthenia Gravis, Parkinson's disease, Muscular Dystrophy, and other central nervous system (CNS) diseases and disorders.

One typical delta opiodergic receptor agonist is DADLE, an endogeneous opioid pentapeptide hormone (Tyr-D-Ala-Gly-Phe-D-Leu) that is known to be useful for inhibiting calcium channel activity via its interaction with the opioidergic receptor, and that is often used as a standard in activity studies of the opioid receptors.

The cells of the invention provide great benefits. They are easy to obtain in large numbers, they can be differentiated into neuron-like cells. Using various tools, such as electrical measurements and biochemical assays, the cells can be easily used to study biochemical signaling mechanisms in these cells. Because they cells of the invention are powerful models of brain-derived neurons, they can be used in a research setting to develop a mechanistic understanding of signaling processes that are aberrant in many of the common neurological defects. They also can be used, as described above, to identify specific compounds that inhibit calcium channel activity in hippocampal neurons, either directly of through interaction with a receptor.

As used herein, the term “voltage gated calcium current activity” is has its accustomed meaning of the inflow of calcium ions from extracellular regions into a cell when the potential difference across the cell membrane is lowered. A description of such voltage-gated activity can be found in Principles of Neural Science, Kandel, E. R., Schwartz, J. H., and Jessel, T. M., Elsevier (third edition), incorporated by reference herein in its entirety for all purposes.

As used herein, the term “hippocampal neuron-derived cell line” is intended to mean an immortalized cell line, for example a hybrid neuroblastoma cell line, that is derived from a hippocampal neuron. One example of such a cell line is HN2, which is commercially available from BTX Molecular Delivery System (MA), Product number PRO546. A further example is HN33 cells. Thus, the term “hippocampal neuron-derived cell” means a cell from such a cell line.

The term “P-type ATPase” has its recognized meaning as the family of ATPases that harbor a DKTG motif in which the Asp (D) residue accepts the γ-phosphate (P) from ATP during the catalytic cycle, forming a covalent acylphosphate intermediate. The term “ATPase II” is intended to have its accustomed meaning of a Mg²⁺-ATPase that is believed to also harbor aminophospholipid translocase (APTL). See Halleck, M. S. et al., Physiol. Genomics, 1 (1999) 139-150; Tang, X., et al., Science, 272 (1996) 1495-1497.

The term “APTase transformed” denotes a cell that has been modified to express an ATPase, for example a P-type ATPase, or an APTase II, by, for example, transfection with an expression vector containing a cDNA for such an APTase.

The term “HN2 cell” denotes the cell commercially available from BTX Molecular Delivery System (MA), Product number PRO546.

The term “inhibit calcium channels” or “inhibition of calcium channels” is intended to mean a decrease in calcium channel activity. Such calcium channel activity can be measured by patch-clamp electrophysiological measurement, as is known in the art. Other suitable methods of measuring calcium channel activity will be apparent to those of skill in the art. The measurement of the ability of a candidate or other compound to inhibit activity of calcium channels can be performed by any such method.

The term “N-type calcium channels” has its accustomed meaning of specific voltage-gated calcium channels that occur mainly on neurons.

The term “calcium channel inhibitor” is intended to mean a compound that inhibits calcium channel activity, as described above.

The term “stimulator of a receptor that inhibits calcium channels” is intended to mean a compound that interacts with a receptor (for example as a ligand) to cause an intracellular response that results in the inhibition of calcium channel activity as described above.

The compounds identified by the methods of the invention and their pharmaceutically acceptable salts are useful in for the treatment of, and/or the alleviation of symptoms of, diseases and disorders that are characterized by or that cause abnormal levels of calcium channel activity. The compounds of the invention can be used alone, or in a pharmaceutical composition containing one or more compounds of the invention, in combination with one or more pharmaceutically acceptable carriers. Thus, in further aspects, the present invention includes pharmaceutical compositions including one or more compounds identified by the methods of the invention, and methods of treating such diseases or disorders, and methods of ameliorating symptoms of such diseases and disorders, that include administering one or more compounds of the invention, preferably with an acceptable pharmaceutical carrier.

The compounds identified by the methods of the invention can be formulated in pharmaceutical compositions that can include one or more compounds of the invention and one or more pharmaceutically acceptable carriers. The compounds of the invention can be administered in powder or crystalline form, in liquid solution, or in suspension. They may be administered by a variety of means known to be efficacious for the administration of antibiotics, including without limitation topically, orally and parenterally by injection (e.g., intravenously or intramuscularly).

When administered by injection, a preferred route of delivery for compounds of the invention is a unit dosage form in ampules, or in multidose containers. The injectable compositions may take such forms as suspensions, solutions, or emulsions in oily or aqueous vehicles, and may contain various formulating agents. Alternatively, the active ingredient may be in powder (lyophillized or non-lyophillized) form for reconstitution at the time of delivery with a suitable vehicle, such as sterile water. In injectable compositions, the carrier is typically comprised of sterile water, saline or another injectable liquid, e.g., peanut oil for intramuscular injections. Also, various buffering agents, preservatives and the like can be included.

Topical applications may be formulated in carriers such as hydrophobic or hydrophilic bases to form ointments, creams, lotions, in aqueous, oleaginous or alcoholic liquids to form paints or in dry diluents to form powders.

Oral compositions may take such forms as tablets, capsules, oral suspensions and oral solutions. The oral compositions may utilize carriers such as conventional formulating agents, and may include sustained release properties as well as rapid delivery forms.

The dosage to be administered depends to a large extent upon the condition and size of the subject being treated, the route and frequency of administration, the sensitivity of the pathogen to the particular compound selected, the virulence of the infection and other factors. Such matters, however, are left to the routine discretion of the physician according to principles of treatment well known in the antibacterial arts. Another factor influencing the precise dosage regimen, apart from the nature of the infection and peculiar identity of the individual being treated, is the molecular weight of the compound.

The compositions for human delivery per unit dosage, whether liquid or solid, may contain from about 0.01% to as high as about 99% of active material, the preferred range being from about 10-60%. The composition will generally contain from about 15 mg to about 2.5 g of the active ingredient; however, in general, it is preferable to employ dosage amounts in the range of from about 250 mg to 1000 mg. In parenteral administration, the unit dosage will typically include the pure compound in sterile water solution or in the form of a soluble powder intended for solution, which can be adjusted to neutral pH and isotonic.

Compounds provided herein can be formulated into pharmaceutical compositions by admixture with pharmaceutically acceptable nontoxic excipients and carriers. As noted above, such compositions may be prepared for use in parenteral administration, particularly in the form of liquid solutions or suspensions; or oral administration, particularly in the form of tablets or capsules; or intranasally, particularly in the form of powders, nasal drops, or aerosols; or dermally, via, for example, transdermal patches; or prepared in other suitable fashions for these and other forms of administration as will be apparent to those skilled in the art.

The composition may conveniently be administered in unit dosage form and may be prepared by any of the methods well known in the pharmaceutical art, for example, as described in Remington's Pharmaceutical Sciences (Mack Pub. Co., Easton, Pa., 1980). Formulations for parenteral administration may contain as common excipients sterile water or saline, polyalkylene glycols such as polyethylene glycol, oils and vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of the active compounds. Other potentially useful parenteral delivery systems for these active compounds include ethylene-vinyl acetate copolymer particles, osmotic pumps, implantable infusion systems, and liposomes. Formulations for inhalation administration contain as excipients, for example, lactose, or may be aqueous solutions containing, for example, polyoxyethylene-9-lauryl ether, glycocholate and deoxycholate, or oily solutions for administration in the form of nasal drops, or as a gel to be applied intranasally. Formulations for parenteral administration may also include glycocholate for buccal administration, a salicylate for rectal administration, or citric acid for vaginal administration. Formulations for transdermal patches are preferably lipophilic emulsions.

The materials of this invention can be employed as the sole active agent in a pharmaceutical or can be used in combination with other active ingredients, e.g., other growth factors which could facilitate neuronal survival or axonal regeneration in diseases or disorders.

The concentrations of the compounds described herein in a therapeutic composition will vary depending upon a number of factors, including the dosage of the drug to be administered, the chemical characteristics (e.g., hydrophobicity) of the compounds employed, and the route of administration. In general terms, the compounds of this invention may be provided in effective inhibitory amounts in an aqueous physiological buffer solution containing about 0.1 to 10% w/v compound for parenteral administration. Typical dose ranges are from about 1 mg/kg to about 1 g/kg of body weight per day; a preferred dose range is from about 0.01 mg/kg to 100 mg/kg of body weight per day. Such formulations typically provide inhibitory amounts of the compound of the invention. The preferred dosage of drug to be administered is likely, however, to depend on such variables as the type and extent of progression of the disease or disorder, the overall health status of the particular patient, the relative biological efficacy of the compound selected, and formulation of the compound excipient, and its route of administration.

While the present invention has been described with specificity in accordance with certain of its preferred embodiments, the following examples serve only to illustrate the invention and are not intended to limit the same.

### EXAMPLES

### Example 1

Preparation of ATPase II-Overexpressing Cell Lines

The ATPase II cDNA sequence in the original vector (pBluescript) (a kind gift from Robert Schlegel) was cleaved by digestion with XbaI and SalI and inserted between XbaI and SalI sites in the mammalian expression vector pCMV6c. The recombinant construct obtained (pCMV-ATPase II) was linearized by digestion at a PvuI site within the β-lactamase gene present on the same vector and then transfected into the hippocampal neuron-derived, hybrid neuroblastoma cell line HN2 by electroporation at 200 V and 975 μF. The transfectants were selected in 400 μg/ml G418 and the clones obtained were screened by Western blotting for ATPase II overexpression. Two clones overexpressing ATPase II (HN2A12, ATTC Accession # PTA-5440 and HN2A22, ATTC Accession # PTA-5441) are discussed herein.

### Example 2

SDS-PAGE and Immunoblot Analysis

The cell or tissue pellets were homogenized on ice in RIPA buffer (PBS containing 1% NP40, 0.5% sodium deoxycholate, 0.1% SDS, 0.5 mM Na₃VO₄ plus freshly added protease inhibitor cocktail; Boeringer) and then assayed for protein concentration. Aliquots containing 150 μg protein or each sample were mixed with an equal volume of SDS-PAGE treatment buffer and incubated on ice for 15-20 min followed by resolution on 7-16% gradient SDS-polyacrylamide gel. The resolved proteins were transferred to nitrocellulose membrane and then probed with an α_{1B} monoclonal antibody. See Scott, V. E. L. et al. (1996) J. Biol. Chem., 271, 3207-3212. The nitrocellulose membrane is blocked overnight (at 4° C.) with 10% milk in 0.1% Tween-PBS, then treated with 1:200 dilution of the α_{1B} antibody in fresh blocking solution, washed three times with Tween-PBS, treated with peroxidase-linked anti-mouse IgG (1:1000), washed, and then the protein bands were detected using the Super Signal kit (Pierce). Following this, the blots were stripped by incubating at room temperature for 1 h in a stripping buffer (0.25 M glycine, pH 2.0), blocked again and then probed with 1:500 dilution of a polyclonal ERK1/2 antibody (Santa Cruz Biotech, Calif.). The secondary antibody concentration maintained in this case was 1:50,000. For the immunodection of ATPase II, a rabbit polyclonal antibody directed toward the N-terminal region was used at 1:10,000 dilution. See Ding, J., et al. (2000) J. Biol. Chem., 275, 23378-23386. The same secondary antibody as used for the immunodetection of ERK1/2 was used also for the detection of ATPase II. See Adayev, T., et al. (2003) Biochim Biophy. Acta, 1640, 85-96.

### Example 3

Electrophysiology

The pipette solution used to measure Ca₂₊ current was composed of (in mM): CsCl (110), HEPES (40), EGTA (10), MgATP (4), MgCl₂ (1), GTP (0.3), phosphocreatine (14) (pH adjusted to 7.3 with CsOH). See Adayev et al., J. Neurochem., 192 (1999) 1489-1496. The extracellular solution was continually perfused at a rate of ab72out 2 ml/minute into a bath containing about 1 ml of recording solution. The external recording solution, designed to isolate calcium channel currents (carried by Ba²⁺), contained the following (in mM): TEACl (140), BaCl₂ (30), HEPES (10), sucrose (20): pH adjusted to 7.3 with TEAOH. The contribution of Na⁺ ions was blocked by the addition of 0.1 mM tetrodotoxin (TTX) to all external solutions. Drugs that dissolve in the extracellular solution were added to the perfusate.

An Axopatch 200A patch clamp amplifier was used to voltage-clamp neurons with truncated dendrites and a cell soma with one dimension of at least 20 μm; using the whole cell configuration. Electrodes, pulled from soda-lime glass capillary tubes ranged in resistance from 1.8-2.5 MOhms. The series resistance circuit of the amplifier was used to compensate 75% of the apparent series resistance. Clamp settling time was typically less than 300 microseconds. When measuring Ca²⁺ currents in TEA, the seal resistance often fell between 1-2 GOhms. During the experiment, at regular periods, we obtained leak sweeps. Leak sweeps consisted of 16 averaged hyperpolarizing steps of 10 mV. The leak sweep currents were subtracted from the individual current records, offline. The data was filtered at 2 KHz then digitized at ˜100 microseconds per point. Voltage protocols were generated and analyzed by an IBM PC Pentium clone using the PClamp6 software and the resultant data was analyzed and prepared using both PClamp6 and SigmaPlot 4.0.

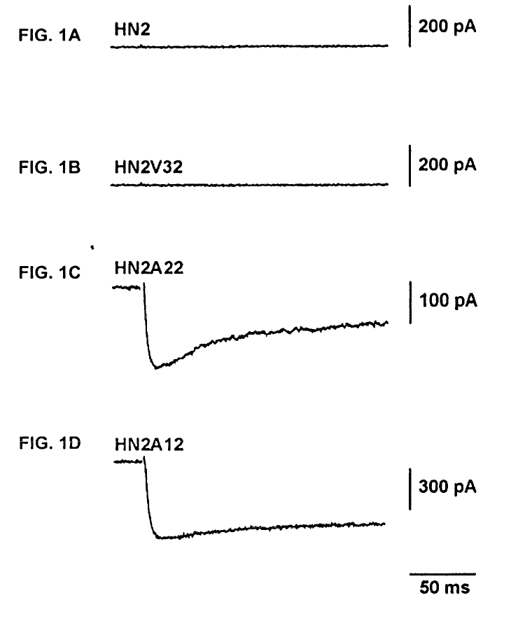

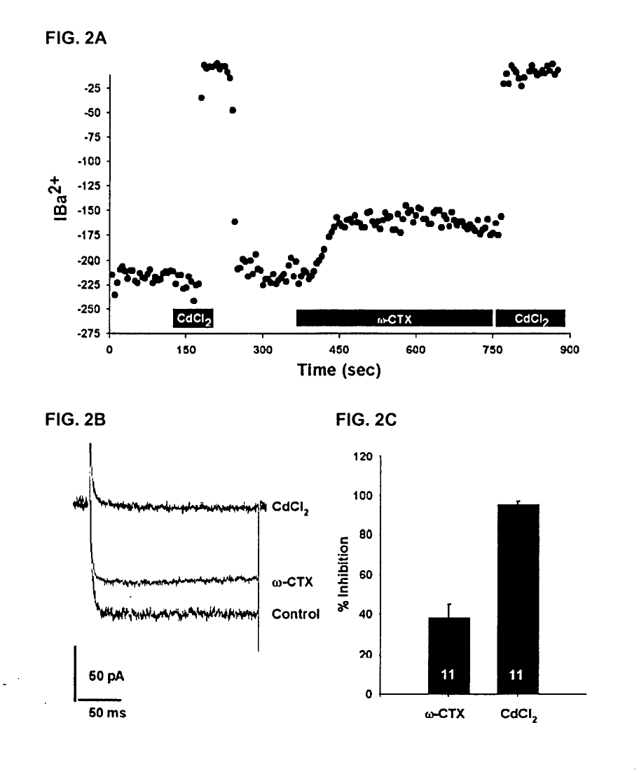

The ATPase II-overexpressing clones displayed significant levels of voltage-gated calcium current, whereas the parent HN2 cells did not. Patch-clamp electrophysiology showed that although the parent HN2 cells did not display any calcium current (FIG. 1a), almost all ATPase II-overexpressing clones display voltage gated calcium current, which was recorded using Ba²⁺ as the charge carrier (FIGS. 1C and D). Further experiments shown in FIGS. 1 and 2 prove that this current was indeed Ca²⁺ current, because it was completely inhibited by CdCl₂ (FIG. 2). Secondly, this Ca²⁺ current was not an artifact of transfection of the vector (pCMV6) used, because another cell line (HN2V32) stably expressing the serotonin 1A receptor from its coding sequence placed in the same vector showed virtually no calcium current (FIG. 1B). A significant part of the calcium current was of the N-type, since the N-type-specific inhibitor, ω-conotoxin-GVIA, blocked about 38% of the current in the HN2A12 cells (FIG. 2). Finally, such mixed calcium current was also observed in the other ATPase II-overexpressing clones, e.g. the HN2A22 clone (FIG. 1C). Among eighty-eight HN2A12 cells tested eighty-five showed mixed Ca²⁺ current (average current 312.9+/−33.7 pA) and out of eight HN2A22 cells tested all displayed the mixed calcium current (average current 143.8+/−9.5 pA).

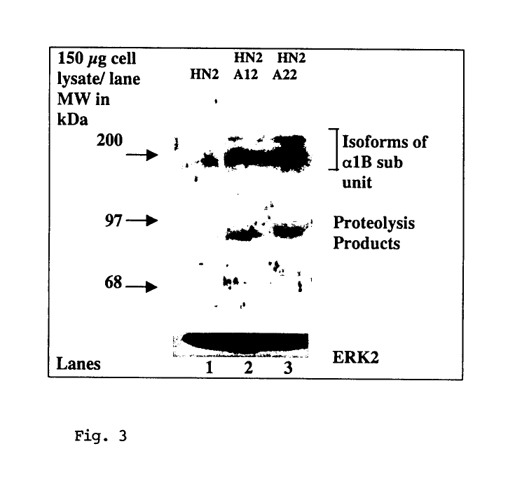

Western blotting shows increased expression of the N-type calcium channel alpha 1B subunit in HN2A12 and HN2A22 cells: The HN2 cells showed a very faint expression of the 210-kDa and 230-kDa isoforms of the α_{1B} subunit of the N-type protein channels complex (FIG. 3, lane 1). In contrast, both the ATPase II-overexpressing clones, HN2A12 and HN2A22, showed dramatically increased expression of the immunoreactive 210 and 230 kDa protein bands (FIG. 3, lanes 2 and 3). Although the HN2 cells showed a faint expression of the α_{1B} subunit, none of our patch-clamp analyses (in the presence of 30 mM Ba²⁺) revealed any calcium current in these cells.

It is intended that each of the patents, applications, and printed publications including catalogs and books mentioned in this patent document be hereby incorporated by reference in their entirety for all purposes.

As those skilled in the art will appreciate, numerous changes and modifications may be made to the preferred embodiments of the invention without departing from the spirit of the invention. It is intended that all such variations fall within the scope of the invention.

What is claimed is:

1. A method for inducing voltage gated calcium current activity in a mammalian hippocampal neuron-derived cell comprising the steps of:
providing a mammalian hippocampal neuron-derived cell; and
transfecting said cell with a recombinant construct comprising a nucleotide sequence encoding a P-type ATPase.

2. The method of claim 1 wherein said P-type ATPase is an APTase II.

3. The method of claim 2 wherein said mammalian hippocampal neuron-derived cell is a murine derived cell.

4. The method of claim 2 wherein said mammalian hippocampal neuron-derived cell is a HN2 cell.

5. The method of claim 2 wherein said recombinant construct comprises a nucleotide sequence encoding a mammalian ATPase II.

6. The method of claim 2 wherein said recombinant construct comprises a nucleotide sequence encoding a murine ATPase II.

7. The method of claim 6 wherein said nucleotide sequence encoding said murine ATPase II comprises the sequence of GeneBank accession number U75321.

8. The method of claim 6 wherein said recombinant construct comprises the expression vector pCMV6c.

9. The method of claim 2 wherein said mammalian hippocampal neuron-derived cell is a HN2 cell; and said recombinant construct comprises a nucleotide sequence encoding a murine ATPase II.

10. The method of claim 6 wherein said nucleotide sequence encoding said murine ATPase II comprises the sequence of GeneBank accession number U75321.

11. A method for identifying a compound that inhibits calcium channels in hippocampal neurons comprising the steps of:
contacting one or more candidate compounds with an ATPase-transformed mammalian hippocampal neuron-derived cell; and
measuring the ability of the compound or compounds to inhibit activity of said channels.

12. The method of claim 11 wherein said ATPase-transformed mammalian hippocampal neuron-derived cell is an ATPase II-transformed mammalian hippocampal neuron-derived cell.

13. The method of claim 12 wherein said ATPase II-transformed mammalian hippocampal neuron-derived cell comprises N-type calcium channel activity.

14. The method of claim 12 wherein said ATPase-transformed mammalian hippocampal neuron-derived cell is a murine derived cell.

15. The method of claim 12 wherein said ATPase-transformed mammalian hippocampal neuron-derived cell is derived from a HN2 cell.

16. The method of claim 12 wherein said ATPase II-transformed mammalian hippocampal neuron-derived cell is produced by a process comprising the steps of:
providing a mammalian hippocampal neuron-derived cell; and
transfecing said cell with a recombinant construct comprising a nucleotide sequence encoding an ATPase II.

17. The method of claim 16 wherein said recombinant construct comprises a nucleotide sequence encoding a mammalian ATPase II.

18. The method of claim 16 wherein said recombinant construct comprises a nucleotide sequence encoding a murine ATPase II.

19. The method of claim 16 wherein said nucleotide sequence encoding said murine ATPase II comprises the sequence of GeneBank accession number U75321.

20. The method of claim 17 wherein said mammalian hippocampal neuron-derived cell is a HN2 cell; and said recombinant construct comprises a nucleotide sequence encoding a murine ATPase II.

21. The method of claim 20 wherein said nucleotide sequence encoding said murine ATPase II comprises the sequence of GeneBank accession number U75321.

22. A method for identifying a compound that stimulates a receptor that inhibits calcium channels in hippocampal neurons comprising the steps of:
contacting one or more candidate compounds with an ATPase transformed mammalian hippocampal neuron-derived cell, wherein said cell expresses at least one receptor that modulates said calcium channels; and
measuring the ability of the compound or compounds to inhibit activity of said channels.

23. The method of claim 22 wherein said ATPase-transformed mammalian hippocampal neuron-derived cell is an ATPase II-transformed mammalian hippocampal neuron-derived cell.

24. The method of claim 23 wherein said ATPase II-transformed mammalian hippocampal neuron-derived cell is derived from a HN2 cell.

25. The method of claim 23 wherein ATPase II-transformed mammalian hippocampal neuron-derived cell is produced by a method comprising the steps of:
providing a mammalian hippocampal neuron-derived cell; and
transfecting said cell with a recombinant construct comprising a nucleotide sequence encoding an ATPase II.

26. The method of claim 25 further comprising the step of:
(c) transfecting said cell with a recombinant construct comprising a nucleotide sequence encoding a receptor that modulates said calcium channels.

27. The method of claim 26 wherein said recombinant construct of said step (c) comprises a nucleotide sequence encoding a receptor selected from purinergic receptors, serotonin 1A receptors, alpha-adrenergic receptors, adenosine receptors, opiodergic receptors and somatostatin receptors.

28. The method of claim 25 wherein said recombinant construct comprises a nucleotide sequence encoding a mammalian ATPase II.

29. The method of claim 25 wherein said recombinant construct comprises a nucleotide sequence encoding a murine ATPase II.

30. The method of claim 25 wherein said nucleotide sequence encoding said murine ATPase II comprises the sequence of GeneBank accession number U75321.

31. The method of claim 28 wherein said recombinant construct comprises the expression vector pCMV6c.

32. The method of claim 25 wherein said mammalian hippocampal neuron-derived cell is a HN2 cell; and said recombinant construct comprises a nucleotide sequence encoding a murine ATPase II.

33. The method of claim 32 wherein said nucleotide sequence encoding said murine ATPase II comprises the sequence of GeneBank accession number U75321.

34. The method of claim 22 wherein said ATPase transformed mammalian hippocampal neuron-derived cell expresses a receptor selected from the group consisting of purinergic receptors, serotonin 1A receptors, alpha-adrenergic receptors, adenosine receptors, opiodergic receptors and somatostatin receptors.

35. An ATPase transformed mammalian hippocampal neuron-derived cell having voltage gated calcium current activity.

36. An ATPase II-transformed mammalian hippocampal neuron-derived cell having voltage gated calcium current activity.

37. The ATPase transformed mammalian hippocampal neuron-derived cell of claim 35 wherein said voltage gated calcium current activity comprises N-type calcium channel activity.

38. The ATPase transformed mammalian hippocampal neuron-derived cell of claim 36 wherein said voltage gated calcium current activity comprises N-type calcium channel activity.

39. An ATPase transformed mammalian hippocampal neuron-derived cell, wherein said cell comprises N-type calcium channels.

40. An ATPase II transformed mammalian hippocampal neuron-derived cell, wherein said cell comprises N-type calcium channels.

41. The ATPase transformed mammalian hippocampal neuron-derived cell of claim 35 wherein said cell is produced by a process comprising transfecting a mammalian hippocampal neuron-derived cell with a recombinant construct comprising a nucleotide sequence encoding an ATPase II.

42. The ATPase transformed mammalian hippocampal neuron-derived cell of claim 41 wherein said mammalian hippocampal neuron-derived cell is a murine derived cell.

43. The ATPase transformed mammalian hippocampal neuron-derived cell of claim 41 wherein said mammalian hippocampal neuron-derived cell is a HN2 cell

44. The ATPase transformed mammalian hippocampal neuron-derived cell of claim 41 wherein said recombinant construct comprises a nucleotide sequence encoding a mammalian ATPase II.

45. ATPase transformed mammalian hippocampal neuron-derived cell of claim 41 wherein said recombinant construct comprises a nucleotide sequence encoding a murine ATPase II.

46. ATPase transformed mammalian hippocampal neuron-derived cell of claim 45 wherein said nucleotide sequence encoding said murine ATPase II comprises the sequence of GeneBank accession number U75321.

47. The ATPase transformed mammalian hippocampal neuron-derived cell of claim 41 wherein said mammalian hippocampal neuron-derived cell is a HN2 cell; and said recombinant construct comprises a nucleotide sequence encoding a murine ATPase II.

48. The ATPase transformed mammalian hippocampal neuron-derived cell of claim 47 wherein said nucleotide sequence encoding said murine ATPase II comprises the sequence of GeneBank accession number U75321.

49. An ATPase II transformed mammalian hippocampal neuron-derived cell having voltage gated calcium current activity produced by a method comprising transfecting a HN2 cell with a recombinant construct comprising an expression vector; said expression vector comprising a nucleotide sequence encoding a murine ATPase II; wherein said nucleotide sequence has the sequence of GeneBank accession number U75321.

50. The cell line HN2A12.

51. The cell line HN2A22.
